(19) United States
(12) Patent Application Publication
(10) Pub. No.: US 2001/0034328 A1
(43) Pub. Date: Oct. 25, 2001

(54) Treatment of male chronic pelvic pain syndrome

(76) Inventor: Stephen LaHue Cartt, San Carlos, CA (US)

Correspondence Address: John A. Dhuey, ALZA Corporation, P.O. Box 7210, Mountain View, CA, 94043-7210, US

(21) Appl. No.: 09/785,816

(22) Filed: Feb. 16, 2001

Related U.S. Application Data
(60) Provisional application No. 60/185,185, filed on Feb. 25, 2000.

(51) Int. Cl. A61K 31/70

(52) U.S. Cl. 514/23

(57) ABSTRACT

Dosage forms and methods for the treatment of symptoms of male chronic pelvic pain syndrome are described.

### CROSS-REFERENCE TO RELATED APPLICATIONS

This application claims the priority of U.S. application Ser. No. 60/185,185, filed Feb. 25, 2000, which is incorporated herein by reference.

### FIELD OF THE INVENTION

This invention pertains to the treatment of male chronic pelvic pain syndrome. In particular, the invention is directed to methods and dosage forms for the treatment of symptoms of male chronic pelvic pain syndrome, including, inter alia, pain, voiding dysfunction and sexual dysfunction, and for the treatment of underlying conditions, such as inflammatory or noninflammatory abacterial prostatitis.

### BACKGROUND OF THE INVENTION

Parenthetical numerals in this section refer to the publications listed at the end of the section unless the context requires otherwise.

Prostatitis is the most common urological diagnosis in men under 50 years of age and the third most common diagnosis in men over 50 years, accounting for more than 2 million office visits per year in the U.S.A. (1). Historically, male chronic pelvic pain syndrome (which is sometimes abbreviated as “CPPS”) has been referred to as abacterial prostatitis and/or prostatodynia. A National Institutes of Health (NIH) Consensus Conference (4) established the current classification of prostatitis in 1995: Category I—Acute bacterial prostatitis; Category II—Chronic bacterial prostatitis; Category III—Chronic pelvic pain syndrome (CPPS); Category IIIA—Inflammatory CPPS; Category IIIB—Noninflammatory CPPS; and Category IV—Asymptomatic inflammatory prostatitis.

Male CPPS (Category III prostatitis) is much more frequent than the better understood bacterial prostatitis (Categories I and II), yet treatment remains elusive. Antibiotics are not routinely indicated and anxiolytics, antispasmodics, anti-inflammatories, alpha blockers, psychological support, reassurance and other conservative measures are usually not helpful (2,3).

Noninfectious inflammation of the lower urinary tract associated with pain and voiding dysfunction is characteristic of chronic pelvic pain syndrome (CPPS) in males. CPPS may be associated with inflammatory and noninflammatory abacterial prostatitis and associated pain, voiding dysfunction, and sexual dysfunction. The condition is poorly understood, has no known definitive etiology, few specific diagnostic criteria and no uniformly successful treatment regimen.

In chronic pelvic pain syndrome, pain may be localized in the suprapubic area, perineum, and external genitalia. Frequency, urgency, nocturia (and sometimes obstruction) are typically the predominant voiding complaints. Ejaculatory and impotence problems in men with prostatitis are frequent complaints (2,8). These symptoms may reduce quality of life and interfere with the patient's ability to work and function to his full capacity.

Wedren (11) reported the results of a blinded pilot trial in which male subjects exhibiting signs and symptoms of Category III prostatitis were treated with 200 mg bid pentosan polysulfate or placebo. After 3 months of treatment, it was reported that there was no evident difference between the treatment group and the placebo group, although the median measurement score for the group treated with pentosan polysulfate was lowered to a greater extent than the placebo group was lowered. It was reported that there appeared to be some improvement in symptoms of myalgia/arthralgia exhibited by the subjects in the treatment group.

Marshall et al. (13) describes a study in which twenty-one terminally ill cancer patients receiving pentosan polysulfate at doses of approximately 900-2100 mg/day for up to 8 months. Twenty patients experienced some degree of bleeding from the rectum or digestive tract.

The following publications, some of which have been referenced above by number, may be referred to for more detailed descriptions of this general area of urology: (1) Collins, M M, Stafford, E A, O'Leary, M P et al. 1997. How common is prostatitis? A national survey of physician visits. J. Urol. 157: 243A; (2) Nickel, J C, Prostatitis: myths and realities (1998). Urology 51: 362-366; (3) Nickel, J C. (1996) Rational management of nonbacterial prostatitis and prostatodynia: Current Opinion in Urology. 6:53-58; (4) Chronic Prostatitis Workshop. Summary Statement. Bethesda Md. National Institute of Health. Dec. 7-8, 1995; (5) Berger, R E, Miller, J E, Rothman, I et. al. Bladder petechiae after cystoscopy and hydrodistention in men diagnosed with prostate pain. 1998; J Urol 159: 83-85; (6) Novicki D E, Larson T R, Swanson S K. Interstitial cystitis in men. Urology 1998; 52: 621-624; (7) Miller J, Rothman I, Bavendam T G, Berger R (1995). Prostatodynia and interstitial cystitis: One and the same? Urology 45:587-590; (8) Simon L J, Landis J R, Erickson D R, Nyberg L M. (1997). The interstitial cystitis data base study: concepts and preliminary baseline descriptive statistics. Urology 49: (Suppl 5A) 64-75; (9) Parsons, C L; Benson, G; Childs, S J et al: A quantitatively controlled method to study prospectively interstitial cystititis and demonstrate the efficacy of pentosan polysulfate. 1993; J Urol 150:845-848; (10) Neal D E Jr, Clejan S, Sarma D, Moon T D Jr. (1992) Prostate specific antigen and prostatitis I. Effect of prostatitis on serum PSA in the human and nonhuman primate. Prostate 20:105-111; (11) Wedren, H: Effects of sodium pentosan polysulfate on symptoms related to chronic nonbacterial prostatitis. Scand. J. Urol Nephrol 1987; 21: 81-88; (12) Litwin M S, McNaughton-Collins M, Fowler F J, Nickel C, Calhoun E A, et al. The NIH chronic prostatitis symptom index (NIH-CPSI): development and validation of a new outcomes measure. J Urology 1999;161(4, Supplement):31; (13) Marshall J L, Wellstein A, Rae J, DeLap R J, Phipps K, Hanfelt J, Yunmbam M K, Sun J X, Duchin K L, Hawkins M J. (1997). Phase I trial of orally administered pentosan polysulfate in patients with advanced cancer. Clinical Cancer Research 3:2347-2354; (14) Nickel J C, Sorenson R. (1994) Transurethral microwave thermotherapy of nonbacterial prostatitis and prostatodynia: Initial experience. Urology 44:458-460; (15) Nickel J C, Sorensen R. (1996) Transurethral microwave thermotherapy for nonbacterial prostatitis: A randomized double-blind sham controlled study using new prostatitis specific assessment questionnaires. J Urol 155: 1950-1954; and (16) O'Leary M P, Fowler F J, Lenderking W R, Barber B, et al. (1995). A brief male sexual function inventory for urology. Urology 46:697-706.

### SUMMARY OF THE INVENTION

In one aspect, the invention comprises a method of treating symptoms of male chronic pelvic pain syndrome in a subject suffering therefrom which comprises orally administering an amount of pentosan polysulfate for a period of time and at a frequency during that period that is effective in alleviating the subject's symptoms. Symptoms may include one or more of the following: pain; voiding dysfunction, such as too-frequent urination and excessive urgency to urinate; and sexual dysfunction, such as impotence and pain upon ejaculation. The pentosan polysulfate may be administered daily for at least one week, two to four weeks, four weeks to eight weeks, eight weeks to sixteen weeks, or for longer periods of time, which may be months or years. Effective amounts of pentosan polysulfate to treat symptoms of male chronic pelvic pain syndrome will be greater than 400 mg/day, and typically may range from 600-1200 mg per day, often at least 900 mg per day. The foregoing doses may be administered initially, and chronic treatment may be continued at the same or lower doses.

In another aspect, the invention comprises a method of treating the symptom of pain of male chronic pelvic pain syndrome in a subject suffering therefrom which comprises orally administering an amount of pentosan polysulfate for a period of time and at a frequency during that period that is effective in alleviating the subject's pain. Painful symptoms may include one or more of the following: prostate pain from inflammatory, abacterial prostatitis; prostate pain from noninflammatory prostatitis; urethral pain, which may be chronic or arise upon urination; bladder pain; and pain upon ejaculation. The pentosan polysulfate may be administered daily for at least one week, two to four weeks, four weeks to eight weeks, eight weeks to sixteen weeks, or for longer periods of time, which may be months or years. Effective amounts of the pentosan polysulfate to treat symptoms of male chronic pelvic pain syndrome will be greater than 400 mg/day, and typically may range from 600-1200 mg per day, often at least 900 mg per day. The foregoing doses may be administered initially, and chronic treatment may be continued at the same or lower doses.

In still another aspect, the invention comprises a method of treating the symptom of urination-urge frequency of male chronic pelvic pain syndrome in a subject suffering therefrom which comprises orally administering an amount of pentosan polysulfate for a period of time and at a frequency during that is effective in alleviating the subject's symptoms. The pentosan polysulfate may be administered daily for at least one week, two to four weeks, four weeks to eight weeks, eight weeks to sixteen weeks, or for longer periods of time, which may be months or years. Effective amounts of the pentosan polysulfate to treat symptoms of male chronic pelvic pain syndrome will be greater than 400 mg/day, and typically may range from 600-1200 mg per day, often at least 900 mg per day. The foregoing doses may be administered initially, and chronic treatment may be continued at the same or lower doses.

In yet another aspect, the invention comprises a method of treating the symptom of urgency of urination of male chronic pelvic pain syndrome in a subject suffering therefrom which comprises orally administering an amount of pentosan polysulfate for a period of time and at a frequency during that period that is effective in alleviating the subject's symptom. The pentosan polysulfate may be administered daily for at least one week, two to four weeks, four weeks to eight weeks, eight weeks to sixteen weeks, or for longer periods of time, which may be months or years. Effective amounts of the pentosan polysulfate to treat symptoms of male chronic pelvic pain syndrome will be greater than 400 mg/day, and typically may range from 600-1200 mg per day, often at least 900 mg per day. The foregoing doses may be administered initially, and chronic treatment may be continued at the same or lower doses.

In another aspect, the invention comprises a method of treating the symptom of impotence of male chronic pain syndrome in a subject suffering therefrom which comprises orally administering an amount of pentosan polysulfate for a period of time and at a frequency during that period that is effective in alleviating the subject's symptom. The pentosan polysulfate may be administered daily for at least one week, two to four weeks, four weeks to eight weeks, eight weeks to sixteen weeks, or for longer periods of time, which may be months or years. Effective amounts of the pentosan polysulfate to treat symptoms of male chronic pelvic pain syndrome will be greater than 400 mg/day, and typically may range from 600-1200 mg per day, often at least 900 mg per day. The foregoing doses may be administered initially, and chronic treatment may be continued at the same or lower doses.

In another aspect, the invention comprises a method of treatment of nonbacterial prostatitis which comprises orally administering to a subject exhibiting symptoms of nonbacterial prostatitis at least 600 mg of pentosan polysulfate sodium within a 24-hour period. The daily dose may be up to 1200 mg, but more typically up to 900 mg. The administration of the drug may repeated daily for at least one week, two to four weeks, four weeks to eight weeks, eight weeks to sixteen weeks, or for longer periods of time, which may be months or years.

In yet another aspect, the invention comprises a method of treating one or more of the symptoms of inflammatory or noninflammatory nonbacterial prostatitis in a subject suffering therefrom that comprises orally administering to the subject a daily dose of at least 600 mg of pentosan polysulfate for a period of time effective to alleviate the symptoms.

In yet another aspect, the invention comprises a method of treating the symptoms of chronic inflammatory or noninflammatory nonbacterial prostatitis in a subject suffering therefrom that comprises orally administering to the subject a daily dose of at least 600 mg of pentosan polysulfate for a period of time to alleviate the symptoms and a follow-on daily dose of pentosan polysulfate of at least 200 mg at least once per week, and often on a continuing, daily basis.

In still another aspect of the invention, the administration of the first dose may precede the administration of the second dose by at least four hours, and the administration of the second dose may precede the administration of the third dose by at least four hours. The method may be practiced by administering the drug to the subject when the subject is in the non-fed mode. In that regard, separate doses of drug are administered at least one hour before a meal or at least two hours after a meal, generally three times per day, around the morning, mid-day and evening meals. The method may be practiced in a manner wherein the daily dose is divided into three separate, equal doses and each such separate dose is administered at independent times during a 24-hour period.

In a further aspect, the invention comprises a unitary dosage form adapted for oral administration comprising at least 300 mg of pentosan polysulfate. The dosage form may be a tablet or a capsule. Optionally, binders and other conventional excipients may be added to the dosage form. The dosage form may comprise at least 300 mg of pentosan sulfate and a pharmaceutically-acceptable binder. The dosage form may comprise 300 mg of pentosan polysulfate and microcrystalline cellulose in a size 0 hard gelatin capsule.

In another aspect, the invention comprises a unitary dosage form adapted for oral administration consisting of at least 300 mg of pentosan polysulfate, microcrystalline cellulose and magnesium stearate.

### DETAILED DESCRIPTION OF THE INVENTION

The present invention is best understood by reference to the following definitions and exemplary disclosure provided herein.

Definitions

By “pentosan polysulfate” is meant a sulfated, semi-synthetic polysaccharide composed of β-D-xylopyranose residues having a molecular weight in the range of 1,500 to 6,000 Daltons and its pharmaceutically-acceptable salts. The compound is described in The Merck Index, Eleventh Edition, Merck & Co, Inc., Rahway, N.J. (1989), pg. 7093, and U.S. Pat. Nos. 5,180,715 and 5,643,892. A preferred pentosan polysulfate is the sodium salt having a molecular weight between 4000 and 6000 Daltons.

By “active agent”, “drug”, or “compound” is meant pentosan polysulfate.

By “dosage form” is meant a pharmaceutical composition or device comprising active agent, the composition or device optionally containing inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like, that are used to manufacture and deliver active pharmaceutical agents.

By “effective amount” or an “amount effective to alleviate” or an “amount effective in alleviating” is meant a dose of drug that is greater than 400 mg/day and when administered to a subject provides recognizable relief from a condition or symptom for which the drug is being administered.

By “effective period of time” is meant a period of time over which an amount of drug is administered to a subject to provide recognizable relief from a condition or symptom for which the drug is being administered.

Pentosan polysulfate is manufactured by bene-Arzneimittel, Geretsried, Germany. An oral formulation of pentosan polysulfate sodium in a 100 mg capsule is currently being marketed by ALZA Corporation, Mountain View, Calif., in the United States under the trademark Elmiron® for administration three times per day for the relief of bladder pain or discomfort associated with interstitial cystitis and in Canada for the initial and maintenance treatment of interstitial cystitis. The oral bioavailability of pentosan polysulfate is approximately 3%. It is believed to be partially metabolized by depolymerization and desulfation reactions and to be eliminated in the urine and feces as both metabolites and unchanged drug.

Male chronic pelvic pain syndrome (CPPS) is characterized by symptoms that may include one or more of the following: pain; voiding dysfunction, such as abnormal frequency of urination and urgency to urinate; and sexual dysfunction, such as impotence and pain upon ejaculation. Such symptoms may derive from conditions that include both inflammatory and noninflammatory chronic abacterial prostatitis and other less well-defined conditions. The method of the present invention provides a therapeutically effective treatment to alleviate the symptoms of male chronic pelvic pain syndrome. The present method comprises orally administering an amount of pentosan polysulfate for a period of time and at a frequency during that period that is effective to alleviate one or more of the subject's symptoms.

The amount of pentosan polysulfate that may be effective for initially alleviating one or more of the symptoms will be greater than 400 mg/day, and more often at least 600 mg/day, administered as a daily dose for a selected period of time. Generally, the daily dose of pentosan polysulfate will not exceed 1200 mg per day. For maintenance therapy, continued dosing of pentosan sulfate at 100 mg per day or greater may be utilized.

Most typically, a dose of drug in the range of 600-900 mg/day may be administered. The daily dose may be administered as three separate doses during the 24-hour period. As presently preferred, 900 mg per day of pentosan polysulfate is administered for alleviation of one or more of the symptoms of chronic pelvic pain syndrome. The 900 mg dose may be administered in three separate doses of 300 mg each. For example, the first 300 mg dose may be administered at least four hours prior to the administration of the second 300 mg dose. The administration of the second 300 mg dose may occur at least four hours prior to the administration of the third 300 mg dose. Administration of the drug may be made to the subject in the non-fed mode. For example, separate doses of drug may be administered within a time period of at least one hour before a meal or at least two hours after a meal, i.e., before or after the morning, mid-day and evening meals, respectively.

Depending on the particular nature and severity of the subject's symptoms, symptoms may be alleviated as early as one week or two weeks after administration of the drug is initiated. More typically, however, the initial dose of the drug will be administered on a daily basis for at least 1 week, more typically for periods of 2-4 weeks, 4-8 weeks, 8-16 weeks, 16-32 weeks or more. The drug may be administered at its initial dose on a daily basis for up to 32 weeks or on a continuing basis thereafter. The clinician may utilize observation and patient feedback to determine progress during such period. In particular circumstances and based on patient response, administration of the drug may be terminated or dose adjustments made during the administration period. For example, if at the end of a 16 week period or 32 week period some alleviation of symptoms is achieved, and it is considered that additional relief may be achieved, then the drug may continue to be administered at the initial daily dose and frequency or a different dose and frequency, e.g., 200-900 mg, for an extended period which may be as long as 32 weeks or more.

If one or more of the symptoms of chronic pelvic pain syndrome is relieved, therapy may be discontinued or, in certain circumstances, it may be continued with a follow-on dose of pentosan polysulfate to serve as maintenance therapy. The follow-on daily dose of drug may be at least 200 mg but generally not more than 900 mg administered at least one day per week, more generally 2-7 times per week, and often on a continuing daily basis. As in the case when the initial dose of drug is administered on a daily basis, it is preferred that the continuation dose of drug, on those days on which it is administered, be administered three times per day in separate, equal doses. The follow-on dose may be administered on an everyday basis in appropriate circumstances. Also, it is preferred that the administration of the separate doses be administered to the subject in the non-fed mode.

While administration of an initial 600-900 mg dose may be undertaken with the existing 100 mg capsules presently marketed by Alza Corporation, it has been discovered that it is possible to prepare a unit dosage form, e.g. capsules and tablets containing 300 mg of pentosan polysulfate sodium, that is suitable for oral administration, provides the desired therapeutic effect when administered as taught herein, and still may be comfortably swallowed by the subject. Capsules may be prepared by the following procedures.

### Preparation

Based on a total batch weight of 100 units, 75% pentosan polysulfate sodium and 24.11% of microcrystalline cellulose NF are mixed in a twin shell blender for 25 minutes. Then 0.89% magnesium stearate, which has been screened through a 20 mesh screen, is added to the shell blender containing the pentosan polysulfate/cellulose blend and the resultant mixture is blended for an additional 5 minutes. The finished blend is encapsulated in size #0 capsules in amounts of 400 mg of the blend using a standard hard capsule filling machine, yielding finished capsules containing 300 mg pentosan sulfate sodium, 96.44 mg microcrystalline cellulose and 3.56 mg magnesium stearate. For example, for a one kilogram batch, 750 grams of pentosan polysulfate sodium and 241.1 grams of microcrystalline cellulose are blended as described above. Then 8.9 grams of magnesium stearate is added to the blend and blended to form a finished one kilogram batch, which is then encapsulated in unit doses of 400 mg total weight in #0 hard gelatin capsules with a conventional capsule filling machine, such as, for example, a Type 8 capsule-filling machine (Parke-Davis, Lilly) or machines such as Zanasi (United Machinery), MG-2 (Supermatic) and Hoefliger & Karg (Bosch).

Alternately, tablets containing 300 mg of pentosan polysulfate may be prepared by conventional tableting methods using appropriate amounts of pharmaceutically-acceptable binders and excipients.

Utilizing capsules prepared as above and 100 mg capsules available from ALZA Corporation as appropriate to meet the daily dosing requirements, male subjects having a clinical diagnosis of chronic pelvic pain syndrome are treated with 600, 700, 800 or 900 mg per day of pentosan polysulfate sodium for up to sixteen weeks, generally 1-2 weeks, 2-4 weeks, 4-8 weeks, 8-16 weeks or up to 32 weeks or more. The daily doses may be administered in multiple doses, generally three times per day with each dose containing equal amounts of pentosan polysulfate. As examples, the daily 600 mg dose may be administered as two 300 mg capsules of pentosan polysulfate as described herein in the morning and evening, or as three 200 mg doses (two 100 mg capsules) in the morning, at mid-day and in the evening. Typically, administration is in the non-fed mode with subjects receiving the doses either at least one hour before a meal or at least two hours after a meal. Daily 900 mg doses may be administered as three 300 mg capsules in the morning, at mid-day and in the evening. As with the 600 mg daily dose, administration typically is in the non-fed mode with subjects receiving the doses either at least one hour before a meal or at least two hours after a meal. Similar arrangements may be made for dosing at the other levels, except that in all cases the individual doses in the morning, at mid-day and in the evening, may not be equal (or dosing may not be three times per day), using combinations of the 100 mg and 300 mg capsules.

When male subjects diagnosed as having male pelvic pain syndrome are treated by the foregoing methods, subjects can experience relief of intensity and frequency of one or more of the following symptoms: pain in the suprapubic area, pain in the perineum, pain in the external genitalia, frequency of urination, urgency of urination, nocturia, ejaculatory pain and impotence.

Treated in the described manner, responders in the subject population may achieve relief of one or more symptoms of male chronic pain syndrome, i.e., pain; voiding dysfunction, such as too-frequent urination and excessive urgency to urinate; and sexual dysfunction, such as impotence and pain upon ejaculation as early as one week after initiation of therapy. More typically, relief may be realized upon continued dosing of drug for two weeks or more as described elsewhere herein.

For chronic conditions, administration of drug may be continued for periods longer than sixteen weeks at a maintenance dose that may be equal to or less than the initial dose. The maintenance dose may be between 200 mg/day and 900 mg/day, often 200 mg/day, 300 mg/day, 400 mg/day, 500 mg/day, 600 mg/day, 700 mg/day, 800 mg/day, or 900 mg/day.

The present invention is described and characterized by one or more of the following technical features and/or characteristics, either alone or in combination with one or more of the other features and characteristics: a method of treating nonbacterial prostatitis which comprises orally administering to a subject suffering therefrom at least 600 mg of pentosan polysulfate sodium within a 24-hour period; a method wherein administration is repeated daily for at least one week; a method wherein administration is repeated daily for at least 16 weeks; a method wherein the daily dose is 600-900 mg; a method wherein the daily dose is 900 mg; a method wherein the daily dose is divided into three separate, equal doses and each separate dose is administered at independent times during a 24-hour period; a method wherein each separate dose is administered at least one hour before or at least two hours after the subject's regular morning, mid-day and evening meals, respectively; a unit dosage form comprising 300 mg of pentosan polysulfate; a dosage form which comprises 300 mg of pentosan polysulfate as a tablet or a capsule; a method of treating one or more of the symptoms of male chronic pelvic pain syndrome in a subject suffering therefrom which comprises orally administering an amount of pentosan polysulfate for a period of time and at a frequency during that period that is effective to alleviate one or more of the subject's symptoms; a method wherein the amount of pentosan polysulfate administered is greater than 400 mg per day; a method wherein the amount of pentosan polysulfate administered is at least 600 mg per day; a method of treating chronic nonbacterial prostatitis in a subject suffering therefrom that comprises orally administering to the subject a daily dose of pentosan polysulfate for a period of time effective to alleviate one or more of the symptoms thereof and a follow-on daily dose of pentosan polysulfate that is equal to or less than the initial dose and administered at least once per week; a method wherein the effective daily dose is at least 600 mg, and the follow-on dose is at least 200 mg but not more than 900 mg administered on a daily basis; a method of treatment which comprises orally administering to a subject exhibiting symptoms of chronic nonbacterial prostatitis 600-1200 mg of pentosan polysulfate within a 24-hour period; a method wherein the total daily amount of pentosan polysulfate is administered in three separate doses; a method the total daily amount of pentosan polysulfate is administered in three separate, equal doses; a method of treating one or more of the symptoms of male chronic pelvic pain syndrome in a subject suffering therefrom which comprises orally administering an amount of pentosan polysulfate for a period of time and at a frequency during that period that is effective in alleviating at least one of the subject's symptoms; a method of treating the symptom of pain of male chronic pelvic pain syndrome in a subject suffering therefrom which comprises orally administering an amount of pentosan polysulfate for a period of time and at a frequency during that period that is effective to alleviate the subject's pain; a method wherein the pain is prostate pain; a method wherein the pain is urethral pain; a method wherein the pain is bladder pain; a method wherein the pain accompanies ejaculation; a method of treating the symptom of urination-urge frequency of male chronic pelvic pain syndrome in a subject suffering therefrom which comprises orally administering an amount of pentosan polysulfate for a period of time and at a frequency during that period that is effective in alleviating the subject's symptom; a method of treating the symptom of urgency of urination of male chronic pelvic pain syndrome in a subject suffering therefrom which comprises orally administering an amount of pentosan polysulfate for a period of time and at a frequency during that period that is effective in alleviating the subject's symptom; a method of treating the symptom of impotence of male chronic pain syndrome in a subject suffering therefrom which comprises orally administering an amount of pentosan polysulfate for a period of time and at a frequency during that period that is effective in alleviating the subject's symptom; a method of treating one or more of the symptoms of inflammatory or noninflammatory nonbacterial prostatitis in a subject suffering therefrom that comprises orally administering to the subject a daily dose of at least 600 mg of pentosan polysulfate; a method of treating one or more of the symptoms of chronic inflammatory or noninflammatory nonbacterial prostatitis in a subject suffering therefrom that comprises orally administering to the subject a daily dose of at least 600 mg of pentosan polysulfate for a period of time effective to alleviate the one or more of the symptoms exhibited by the subject and a follow-on daily dose of pentosan polysulfate of at least 200 mg and at least once per week.

The above-described exemplary embodiments are intended to be illustrative in all respects, rather than restrictive, of the present invention. Thus, the present invention is capable of many variations in detailed implementation that can be derived from the description contained herein by a person skilled in the art. All such variations and modifications are considered to be within the scope and spirit of the present invention as defined by the following claims.

What is claimed is:

1. A method of treating nonbacterial prostatitis which comprises orally administering to a subject suffering therefrom at least 600 mg of pentosan polysulfate sodium within a 24-hour period.

2. The method of claim 1 wherein administration is repeated daily for at least one week.

3. The method of claim 1 wherein administration is repeated daily for at least 16 weeks.

4. The method of claim 3 wherein the daily dose is 600-900 mg.

5. The method of claim 4 wherein the daily dose is 900 mg.

6. The method of claim 4 wherein the daily dose is divided into three separate, equal doses and each separate dose is administered at independent times during a 24-hour period.

7. The method of claim 6 wherein each separate dose is administered at least one hour before or at least two hours after the subject's regular morning, mid-day and evening meals, respectively.

8. A unitary dosage form comprising 300 mg of pentosan polysulfate.

9. The dosage form of claim 8 which comprises a tablet or a capsule.

10. A method of treating one or more of the symptoms of male chronic pelvic pain syndrome in a subject suffering therefrom which comprises orally administering an amount of pentosan polysulfate for a period of time and at a frequency during that period that is effective to alleviate one or more of the subject's symptoms.

11. The method of claim 10 wherein pentosan polysulfate is administered daily for at least one week.

12. The method of claim 10 wherein pentosan polysulfate is administered daily for at least 16 weeks.

13. The method of claim 10 wherein the amount of pentosan polysulfate administered is at least 600 mg per day.

14. A method of treating chronic nonbacterial prostatitis in a subject suffering therefrom that comprises orally administering to the subject a daily dose pentosan polysulfate for a period of time effective to alleviate one or more of the symptoms thereof and a follow-on daily dose of pentosan polysulfate that is equal to or less than the initial dose and administered at least once per week.

15. The method of claim 14 wherein the daily dose is at least 600 mg, and the follow-on dose is at least 200 mg but not more than 900 mg administered on a daily basis.

16. A method of treatment which comprises orally administering to a subject exhibiting symptoms of chronic nonbacterial prostatitis 600-1200 mg of pentosan polysulfate sodium within a 24-hour period.

17. The method of claim 16 wherein the total amount of pentosan polysulfate is administered in three separate doses.

18. The method of claim 17 wherein the total amount of pentosan polysulfate is administered in three separate, equal doses.

19. The method of claim 17 wherein the doses of pentosan polysulfate are administered at least one hour before or at least two hours after the subject's morning, mid-day and evening meals, respectively.

20. A method of treating one or more of the symptoms of male chronic pelvic pain syndrome in a subject suffering therefrom which comprises orally administering an amount of pentosan polysulfate for a period of time and at a frequency during that period that is effective in alleviating at least one of the subject's symptoms.

21. A method of treating the symptom of pain of male chronic pelvic pain syndrome in a subject suffering therefrom which comprises orally administering an amount of pentosan polysulfate for a period of time and at a frequency during that period that is effective to alleviate the subject's pain.

22. The method of claim 21 wherein the pain is prostate pain.

23. The method of claim 21 wherein the pain is urethral pain.

24. The method of claim 21 wherein the pain is bladder pain.

25. The method of claim 21 wherein the pain accompanies ejaculation.

26. A method of treating the symptom of urination-urge frequency of male chronic pelvic pain syndrome in a subject suffering therefrom which comprises orally administering an amount of pentosan polysulfate for a period of time and at a frequency during that period that is effective in alleviating the subject's symptom.

27. A method of treating the symptom of urgency of urination of male chronic pelvic pain syndrome in a subject suffering therefrom which comprises orally administering an amount of pentosan polysulfate for a period of time and at a frequency during that period that is effective in alleviating the subject's symptom.

28. A method of treating the symptom of impotence of male chronic pain syndrome in a subject suffering therefrom which comprises orally administering an amount of pentosan polysulfate for a period of time and at a frequency during that period that is effective in alleviating the subject's symptom.

29. A method of treating one or more of the symptoms of inflammatory and noninflammatory nonbacterial prostatitis in a subject suffering therefrom that comprises orally administering to the subject a daily dose of at least 600 mg of pentosan polysulfate.

30. A method of treating one or more of the symptoms of chronic inflammatory and noninflammatory nonbacterial prostatitis in a subject suffering therefrom that comprises orally administering to the subject a daily dose of at least 600 mg of pentosan polysulfate for a period of time effective to alleviate one or more of the symptoms exhibited by the subject and a follow-on daily dose of pentosan polysulfate of at least 200 mg and at least once per week.
